(19) United States
(12) Patent Application Publication
(10) Pub. No.: US 2024/0411046 A1
(43) Pub. Date: Dec. 12, 2024

(54) GENERATING A VIRTUAL MODEL OF A SUBTERRANEAN REGION

(71) Applicant: SAUDI ARABIAN OIL COMPANY, Dhahran (SA)

(72) Inventor: Ali Ahmed Al-Ghamdi, Dammam (SA)

(73) Assignee: SAUDI ARABIAN OIL COMPANY, Dhahran (SA)

(21) Appl. No.: 18/332,700

(22) Filed: Jun. 9, 2023

(51) Int. Cl. G01V 99/00; E21B 44/00; E21B 49/02; G01N 33/24; G06N 20/00

(52) U.S. Cl. CPC G01V 20/00; E21B 44/00; E21B 49/02; G01N 33/24; G06N 20/00; E21B 2200/20

(57) ABSTRACT

A method and system of generating a virtual model of a subterranean region is disclosed. The method may include obtaining characteristics of a plurality of sample points within a first wellbore traversing a subterranean region and for each sample point within the plurality of sample points, assigning a geological category based, at least in part, on the characteristics of the sample point, and assigning a petrophysical category based, at least in part, on the characteristics of the sample point. The method may further include assigning a hybrid category based, at least in part, on the geological category and the petrophysical category and generating the virtual model of a subterranean region of interest based, at least in part, on the hybrid category assigned to each sample point in the plurality of sample points.

### BACKGROUND

The exploration of hydrocarbon resources is influenced by the expected spatial distribution of rock types that specifies oil-in-place and fluid flow. For carbonate rock typing, two distinctive methodologies have been developed to be used in a three-dimensional geological model: a rock typing based on geological characteristics and a rock typing based on petrophysical characteristics. While the geological rock-typing is a roadmap to the localization of depositional environments and their special distribution, the geological rock-typing alone is inapt to guide geoscientists and engineers to understand the petrophysical properties of any reservoir rocks. The petrophysical rock-typing has been required for exploration of reservoir hydrocarbon and field development. However, the coexistence of two methodologies without integration inside a three-dimensional model cannot provide a long-desired virtual model of carbonate reservoir, which achieves a three-dimensional distribution of a different rock types and smooth transitions between different rock types. Accordingly, there exists a need for a method and a system of creating a virtual model of a subterranean region that guides wellbore planning and illustrates carbonate rock typing in a readily executable format.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method of generating a virtual model of a subterranean region. The method may include obtaining characteristics of a plurality of sample points within a first wellbore traversing a subterranean region and for each sample point within the plurality of sample points, assigning a geological category based, at least in part, on the characteristics of the sample point; and assigning a petrophysical category based, at least in part, on the characteristics of the sample point; assigning a hybrid category based, at least in part, on the geological category and the petrophysical category; and generating the virtual model of a subterranean region of interest based, at least in part, on the hybrid category assigned to each sample point in the plurality of sample points.

In another aspect, embodiments disclosed herein relate to a system of generating a virtual model of a subterranean region. The system may include a processor in data communication with a logging unit of a first wellbore, a display connected to the processor, and memory connected to the processor and to the logging unit. The logging unit may obtain characteristics of a plurality of sample points within the first wellbore traversing a subterranean region. By retrieving data about the subterranean region stored in a memory and measurements recorded at the logging unit, the processor may for each sample point within the plurality of sample points, assign a geological category based, at least in part, on the characteristics, assign a petrophysical category based, at least in part, on the characteristics, assign a hybrid category based, at least in part, on the geological category and the petrophysical category, generate the virtual model of a subterranean region for rock typing based, at least in part, on the hybrid category assigned to the each sample point within the plurality of sample points. The display three-dimensionally displays the virtual model of a subterranean region for rock typing, wherein the logging unit records the measurements about the plurality of sample points and the first wellbore in a well log.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn are not necessarily intended to convey any information regarding the actual shape of the particular elements and have been solely selected for ease of recognition in the drawing.

FIG. 1 shows a schematic perspective view of a reservoir region in which a system of generating a virtual model of a subterranean region is implemented in accordance with one or more embodiments.

FIG. 2A shows a schematic perspective view of a geological region in accordance with one or more embodiments.

FIG. 2B shows a schematic vertical view of a geological region in accordance with one or more embodiments.

FIG. 3 shows a graph indicating characteristics of a plurality of sample points within a wellbore, in accordance with one or more embodiments.

FIG. 4 shows a graph indicating characteristics of a plurality of sample points within a wellbore, in accordance with one or more embodiments.

FIG. 5A shows a diagram illustrating the difference between Traditional Dunham Classification and Modified Dunham Classification in accordance with one or more embodiments.

FIG. 5B shows a graph indicating the collection of rock characteristics of a plurality of sample points within a wellbore in accordance with one or more embodiments

FIG. 5C shows a table illustrating a system of generating a virtual model of a subterranean region in accordance with one or more embodiments.

FIG. 6A shows a block diagram of a system of generating a virtual model of a subterranean region in accordance with one or more embodiments.

FIG. 6B shows a schematic view of a system of generating a virtual model of a subterranean region in accordance with one or more embodiments.

FIG. 7 shows a schematic diagram of a neural network in accordance with one or more embodiments.

FIG. 8 shows a schematic diagram of a system of generating a geological model in accordance with one or more embodiments.

FIG. 9 shows a schematic diagram illustrating a system of generating a virtual model of a subterranean region in accordance with one or more embodiments.

FIGS. 10A to 10C show flowcharts describing a method of generating a virtual mode of a subterranean region in accordance with one or more embodiments.

FIG. 11 shows a block diagram of a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (for example, first, second, third) may be used as an adjective for an element (that is, any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments disclosed herein relate to a method and system of generating a virtual model of a subterranean region for rock typing that combines a geological rock typing and a petrophysical rock typing and assigns a hybrid category to various points in the subterranean region. In another aspect, embodiments disclosed herein relate to a method and system of generating a virtual model of a subterranean region by creating a prediction model of a hybrid category on machine learning architecture and by determining a geological category of uncored samples using a classification analysis.

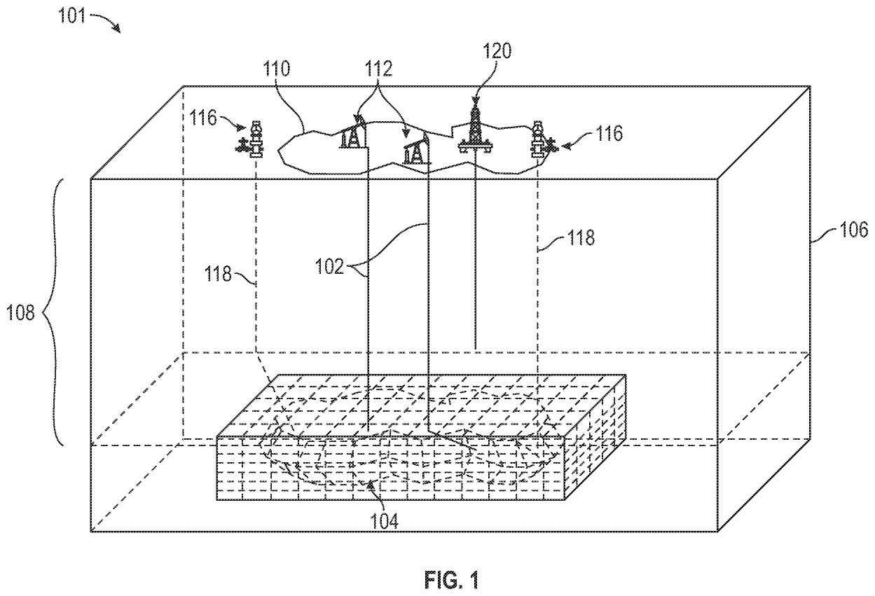

Referring to FIG. 1, FIG. 1 shows a schematic diagram of a reservoir region 101 in which a system of generating a virtual model of a subterranean region is implemented.

As illustrated in FIG. 1, a territory may include one or more reservoir regions (e.g., reservoir region 101) with various production wells (e.g., production wells 102). Likewise, the reservoir region 101 may also include one or more injection wells (e.g., injection well 118) that include functionality for enhancing production by one or more neighboring production wells. The reservoir region 101 may comprise production wells 102 formed from a multiplicity of subterranean layers which contain hydrocarbon deposits and unconventional reservoirs. Information of the subterranean layers around the established/contemplated production well 102 is essential for estimating oil production from each layer.

Porous and permeable rocks are likely to produce more hydrocarbons. Some portions of the rocks contained in a subterranean region are more porous and permeable then others. Accordingly, a production well construction targets these portions in order to reach productive rocks when drilling a subsurface reservoir. For the purpose of hydrocarbon production, mapping the porosity and permeability within the reservoir prior to drilling is therefore important, targeting both production wells, and injection wells (to inject water to sweep the hydrocarbons towards the production wells).

Dunham Classification (1962) is a classification standard for carbonate rocks focused on the depositional texture and composition of rock samples. Under Dunham Classification, observers identify the following features of carbonate rocks: (1) presence or absence of carbonate mud (particles less than 20 microns); (2) abundance of carbonate grains (particles larger than 20 microns); (3) whether the grains are mud supported or grain supported; and (4) evidence of organic binding during deposition. The classification proceeds by observing textures and grainsize of rocks and finding microbial and structural differences. Dunham Classification usually categorizes rocks into groups of grainstone, packstone, wackestone, mudstone and others.

Typically in Dunham Classification, rock samples containing less than 10% grains are included in the mudstone class if they are mud-supported, while rock samples containing more than 10% grains are labeled as wackestone. If rock samples contain mud but are grain-supported and not mud-supported, they are grouped into packstone. If rock samples lack mud and is grain-supported, they are categorized as grainstone. Rock samples whose original components are bound together are assigned to boundstone. Crystalline is the class of rock samples whose depositional textures are not recognizable.

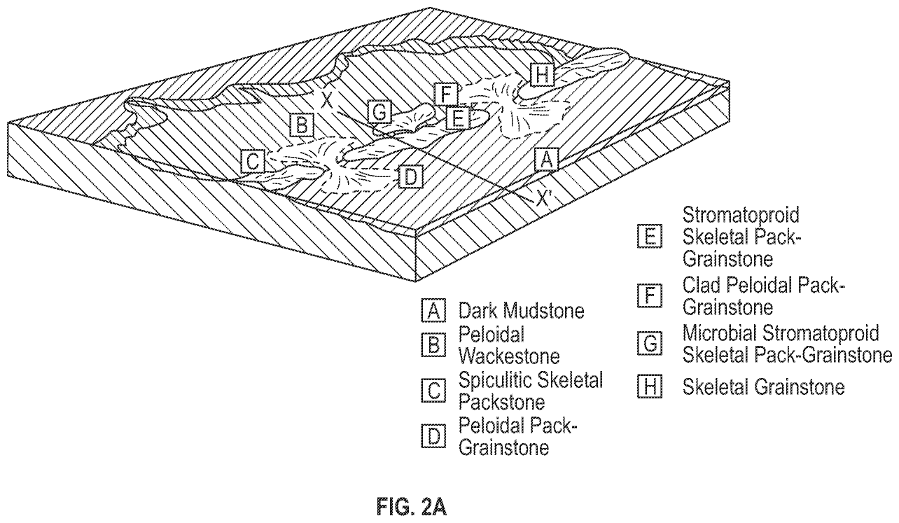

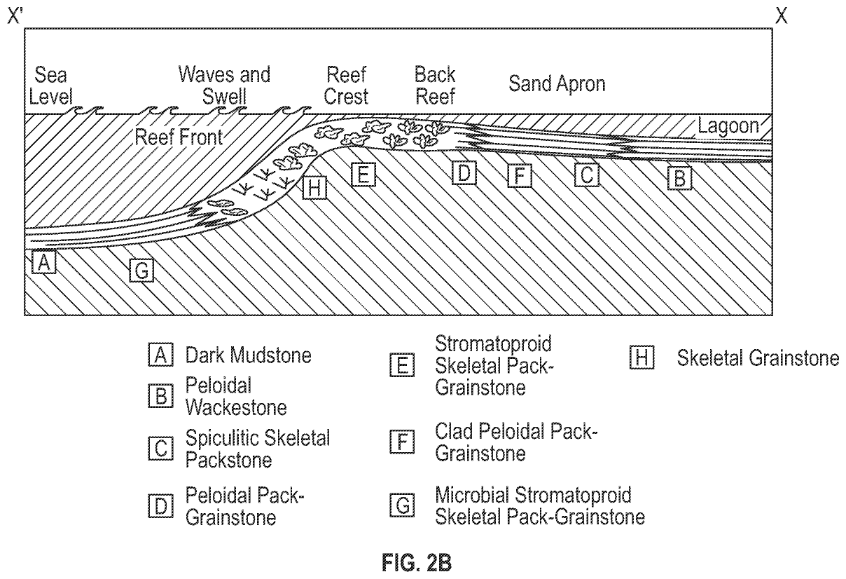

FIG. 2A shows a schematic perspective view, and FIG. 2B shows a schematic vertical view of an example geological region in accordance with one or more embodiments.

More specifically, FIGS. 2A and 2B illustrate the way geological locations and surrounding environment of carbonate rocks are weaved into Dunham Classification. Dunham Classification, as a geological classification, considers the spatial distribution of various carbonate rocks. For example, the grainsize can often be related to the wave and current energy of the water in which the rock forming sediment was deposited: mudstone in calm lagoon water, grainstone in rough (energetic) ocean waves and currents. Understanding this correlation, makes interpolating or extrapolating between wellbores more robust. If a reef can be tracked on seismic data, the reef may be expected to be boundstone, with mudstone on the lagoon side and grainstone on the ocean side.

Traditional Dunham Classification considers textures and grainsizes of rocks as well as microbial and structural differences. In applying Traditional Dunham Classification, an investigator may retrieve preexisting geological databases if geological characteristics of sample points are available. In one instance, the representative reservoir region 101 may have eight geological categories of rocks as a result of the geological/dispositional examination. These 8 categories may include: Group A (dark mudstone); Group B (peloidal wackestone); Group C (spiculitic skeletal packstone); Group D (peroidal pack-grainstone); Group E (stromatoproid skeletal pack-grainstone); Group F (clad peloidal pack-grainstone); Group G (microbial stromatoproid skeletal pack-grainstone); and Group H (skeletal grainstone).

In the example, Dunham Rock Type (DRT) may be assigned as follows: Group A to DRT 0, Group B to DRT 1, Group C to DRT 3, Group D to DRT 4, Group E to DRT 4. Group F to DRT 5, Group G to DRT 5, and Group H to DRT 6. It is not common that most subterranean layers have characteristics corresponding to grainstone rocks and are assigned to one or two identical classes under Traditional Dunham Classification.

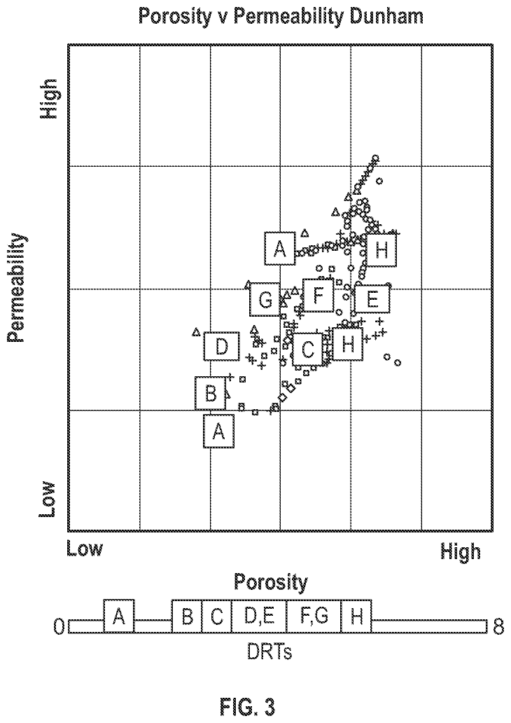

Turning to FIG. 3, FIG. 3 shows example petrophysical characteristics of rock samples obtained from a plurality of sample points in the reservoir region 101. More specifically, FIG. 3 shows petrophysical characteristics of rock samples from the plurality of sample points whose geological classes are assigned to DRT 0 to DRT 8, according to Traditional Dunham Classification. The petrophysical characteristics of rock samples in a particular DRT do not coincide with each other well and spread apart widely on the graph, even if all the sample points are in the same DRT class.

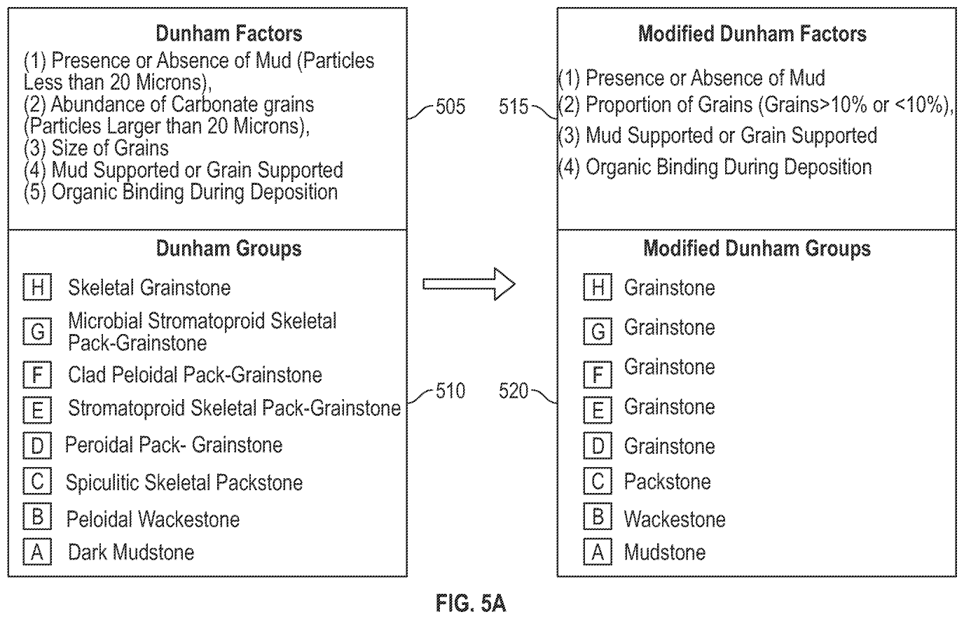

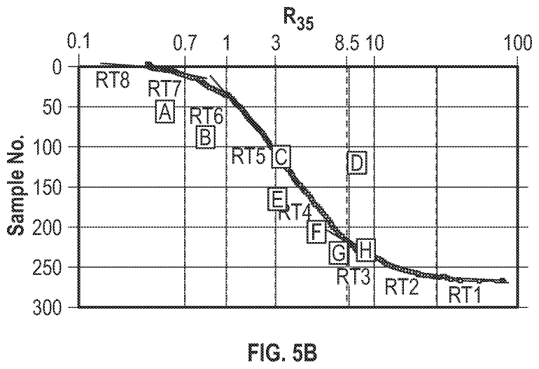

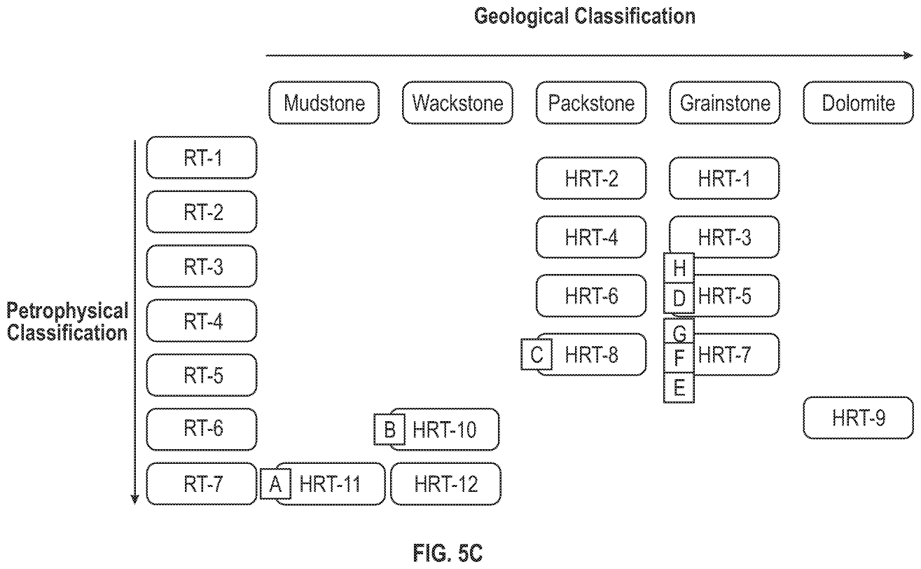

The observed discrepancy between the petrophysical characteristics of the rock samples and their geological classes may be attributed to the following factors: geological classification systems do not incorporate features relevant to permeability (vugs, fractures, intraparticle porosities, and cement elements) even though rocks in the same geological class have been found to have wide varieties of petrophysical properties, from low porosity and permeability to high quality; actual or estimated porosity and/or permeability of rocks do not become a part of geological classifications (as discussed later in relation to FIG. 5A); and regardless of whether packstone, mudstone, and dolomite have similar petrophysical properties, they are assigned to three distinct rock types under Traditional Dunham Classification. As such, there is no direct relationship between a specific DRT and the porosity and permeability measurements of rock samples.

In contrast, petrophysical characteristics provide clues as to the estimated amount of production in cored wells. In particular, the assessment of pore sizes and/or pore throat sizes leads to a better prediction of flow units and matrix permeability. The permeability (y)-porosity (x) characteristics for the plurality of sample points are determinable by mercury injection capillary pressure (MICP) measurements, in accordance with one or more embodiments. The geological rock typing, therefore, is unlikely to have a direct relationship to the quality of rocks. If a rock typing closely matches petrophysical characteristics of the rocks contained in subterranean layers, a wellbore establishment and control can be guided by the rock typing.

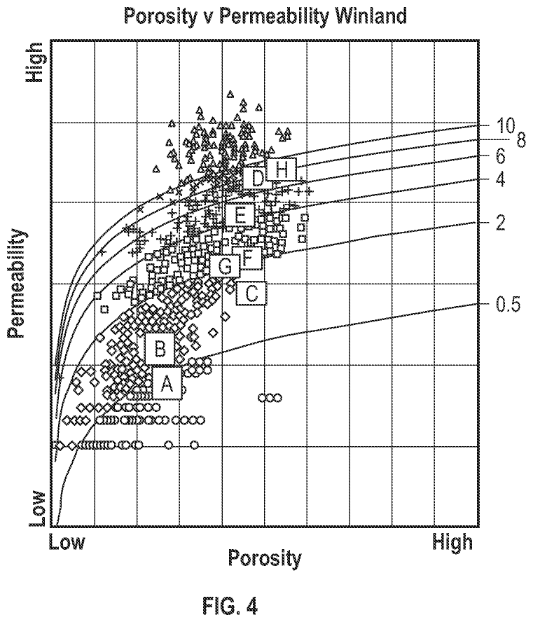

FIG. 4 shows example petrophysical characteristics of rock samples from the plurality of sample points whose petrophysical rock types are assigned to PRT 1 to PRT 7, depending on the size of a calculated pore throat radius according to the Winland R₃₅ method. The Winland R₃₅ method is based on the pore throat radius corresponding to 35% of mercury (non-wetting phase) saturation in mercury injection capillary pressure (MICP) measurements as an indicate of effective flow. Points corresponding to certain R₃₅ values (the pore throat radius corresponding to 35% of mercury saturation=0.5, 2, 4, 6, 8, 10) are shown in curves in various colors. Group D and Group H are close to the curve R₃₅=8, while Group C, Group E, Group F, and Group G are close to the line R₃₅=2. In this example, the plotting of permeability (y)-porosity (x) characteristics of the rock samples align closely with the petrophysical classes (petrophysical category is shown by the color of each sample point). Therefore, the use of at least one petrophysical rock typing such as Winland R₃₅ is useful for operators of wellbores and petrochemical engineers since the availability and accessibility of natural resources in the production well 102 should ideally be evaluated.

Among geological rock types, groups falling into a broad “grainstone” category (Group D. Group E. Group F. Group G, and Group H), are important sources of carbonates and have diverse petrophysical attributes, as shown in FIGS. 3 and 4. Moreover, rock samples belonging to different geological categories may have similar petrophysical characteristics (the similar characteristics observed in Group D and Group H. or the similar characteristics observed among Group C. Group E, Group F, and Group G). Accordingly, if a rock-typing standard places more weight on the petrophysical characteristics and does not consider certain geological characteristics collectable only through specialized examinations (required under Traditional Dunham Classification), such standard obviates time-consuming examinations unrelated to successful oil and gas production.

As such, Modified Dunham Classification may be devised to streamline Traditional Dunham Classification to satisfy practical and economical needs.

FIG. 5A describes one implementation of Modified Dunham Classification, contrasting its factors and groups with those of Traditional Dunham Classification. The left column of the diagram illustrates Traditional Dunham Classification, and the right column illustrates Modified Dunham Classification.

In one or more embodiments, Modified Dunham Classification may utilize the following factors 515 when grouping rock samples: (1) presence or absence of carbonate mud; (2) proportion of grains (grains>10% or <10%); (3) mud-supported or grain-supported; and (4) organic binding during deposition. On the other hand, Traditional Dunham Classification utilizes the following factors 505: (1) presence or absence of carbonate mud (particles less than 20 microns); (2) abundance of carbonate grains (particles larger than 20 microns), (3) size of grains (5 mm to 1 m); (4) mud-supported or grain-supported; and (5) organic binding during deposition. The reduction of classification factors may significantly reduce loss of operational time, associated costs, and an inaccurate classification. Efficiency and accuracy may increase because Modified Dunham Classification groups 520 are more streamlined than Traditional Dunham Classification groups 510, in accordance with one or more embodiments.

Turning to FIG. 5B, FIG. 5B depicts a graph of petrophysical characteristics of the plurality of sample points within the wellbore 601 in relation to sample numbers, in accordance with one or more embodiments. For instance, the petrophysical characteristics are measured by R₃₅ when a petrophysical analyzer 640 performs a petrophysical test on core samples. The obtained data may be submitted to a server, and a processor 650 may calculate R₃₅ according to Winland R₃₅ method. In this example, the processor 650 determines that there should be eight petrophysical categories (RT1 to RT8) based on the distribution of R₃₅.

In one or more examples, Group A (DRT 0) is assigned to RT7, Group B (DRT 1) is assigned to RT6, Group C (DRT 3) is assigned to RT4, Group D (DRT 4) is assigned to RT3, Group E (DRT 4) is assigned to RT4, Group F (DRT 5) is assigned to RT4, Group G (DRT 5) is assigned to RT4, and Group H (DRT 6) is assigned to RT3.

FIG. 5C shows a table illustrating a system of generating a virtual model of a subterranean region in accordance with one or more embodiments. As clear from FIG. 5C, the system integrates the geological category under Modified Dunham Classification and the petrophysical category under Winland R₃₅ method and assigns a hybrid category to each of the subterranean points.

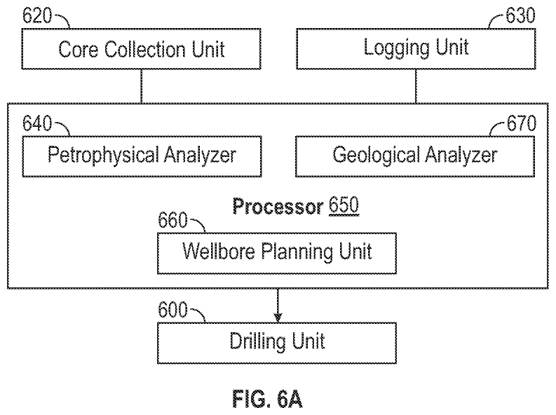

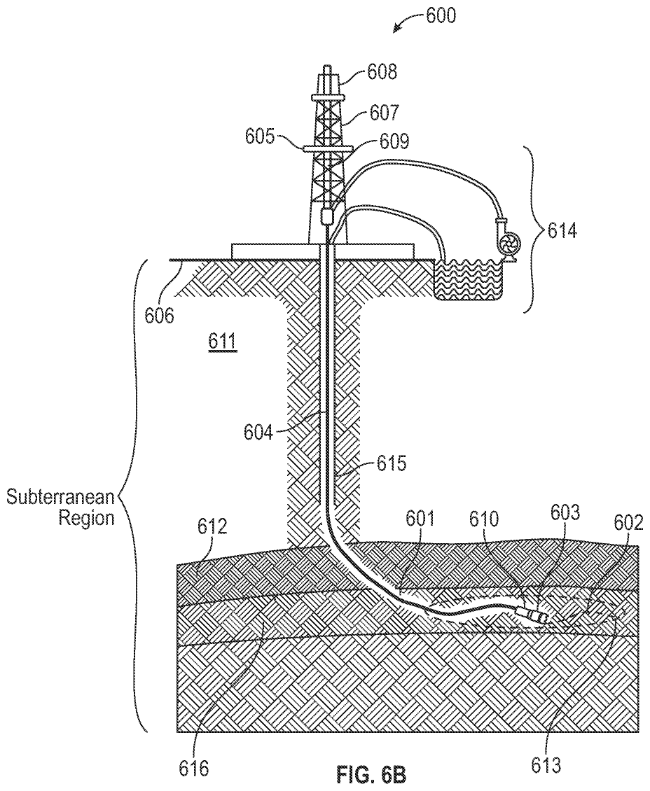

FIG. 6A shows a block diagram of the system of generating a virtual model of a subterranean region in accordance with one or more embodiments. FIG. 6A includes a processor with analyzers (640, 670) and a wellbore planning unit (660), operatively connected to a drilling unit (600), a core collection unit (620) and a logging unit (630). FIG. 6B shows a drilling unit 600 that drills a well 102 as guided by the generated virtual model of subterranean region, ultimately constituting a type of the wellbore plan, in accordance with one or more embodiments.

In some examples, the processor 650 is programmed to determine a hybrid category of each of the sample points after assigning a geological category and a petrophysical category to each of the sample points.

In some embodiments, the determination is based, at least in part, on the geological category and the petrophysical category of the sample points. In this example, Group D's sample points assigned to “Grainstone” (originally peroidal pack-grainstone) whose R₃₅ is no less than 8.5 and less than 10 (“10>R₃₅≥8.5”) are assigned to HRT 5, and Group H's sample points assigned to “Grainstone” (originally skeletal grainstone) whose R₃₅ is 10>R₃₅≥8.5 are also assigned to HRT 5. Groups E, F, G's sample points assigned to “Grainstone” (originally stromatoproid skeletal pack-grainstone, clad peloidal pack-grainstone, and microbial stromatoproid skeletal pack-grainstone) whose R₃₅ is 8.5>R₃₅≥3 are all assigned to HRT 7. In contrast, Group C's sample points assigned to “Packstone” (spiculitic skeletal packstone, originally) whose R₃₅ is 8.5>R₃₅≥3 are assigned to HRT 8.

As such, the processor 650, in assigning a hybrid category to the sample points, considers the geological category of Modified Dunham Classification 520, not Traditional Dunham Classification 510. Additionally, the processor 650 assigns a hybrid category to the sample points, in consideration of the petrophysical category, for example, determined by Winland R₃₅ method. One such example is described by G. W. Gunter, et al., Early Determination of Reservoir Flow Units Using an Integrated Petrophysical Method SPE (Society of Petroleum Engineers) 38679 (1997). The hybrid category integrates the geological category, modified Dunham Classification, with the petrophysical category so that a three-dimensional distribution of predicted rock properties may be generated.

In implementing a system of generating a virtual model of a subterranean region, the processor 650 of the system may obtain aforementioned geological characteristics of the plurality of sample points. In such implementations, a logging unit 630 may obtain characteristics of the plurality of sample points along a subterranean region of the production well 102. For example, the rock density, the seismic velocity, and the resistivity of rock samples may be measured along the segments. Alternatively, data from previously performed remote sensing geophysical surveys, such as seismic surveys, gravity surveys, and active and passive source resistivity surveys may be obtained.

In one example system, a core collection unit 620 obtains core samples from some of the plurality of sample points, which may be spread along a wellbore 601.

In some embodiments, a petrophysical analyzer 640 may perform a petrophysical analysis on the core samples. In other embodiments, the obtained core samples may be examined to extract its geological attributes by a geological analyzer 670.

In some example operations, the processor 650 may receive petrophysical characteristics such as pore radius and permeability and/or geological attributes of the subterranean points from a server (not shown) connected to the processor 650.

Continuing with FIG. 6A, and referring also to FIG. 6B, the processor 650 may include a wellbore planning unit 660 and is configured to determine a drilling target based, at least in part, on the virtual model of a subterranean region, and plan, a planned trajectory 602 of the wellbore 601 to intersect the drilling target 613. In such embodiments, the planned wellbore trajectory 602 is created by the drilling unit 600 to guide drilling of the wellbore 601.

In forming the virtual model of subterranean region, it is helpful to understand the geological processes that form rocks constituting the reservoir region 101 in addition to the information gathering using wellbore measurements, core samples, and remote data sensing, such as seismic surveys, surface resistivity surveys, and gravity surveys. In some embodiments, the system may be located over a formation within the reservoir region 101. The formation may constitute a part of a subterranean region under the production well 102.

In one or more implementations, the system may include the logging unit 630 of the production well 102, a server, a processor 650, a display, and a memory 1106. For example, the system may include hardware and software with functionality for generating a virtual model of a subterranean region and/or performing drilling of a well in accordance with one or more embodiments.

The logging unit 630, which may include logging tools, measures and collects characteristics of a plurality of sample points within the wellbore 601 traversing the subterranean region in some embodiments. As a geological examination, the obtained core samples may be visually examined to extract its geological attributes. The processor 650, in data communication with the logging unit 630, retrieves characteristics of the plurality of sample points that the logging unit 630 measures in some implementations.

In some implementations, the porosity, the permeability, the density, the seismic velocity, and the resistivity of rock samples may be measured in the wellbore 601. In one example, when a core collection unit 620 obtains core samples from some of the plurality of sample points, which may be spread along a wellbore 601, a petrophysical analyzer 640 performs a petrophysical analysis on the core samples.

Additionally, in one or more embodiments, the processor 650 connected with the memory 1106, retrieves characteristics (extraction from geological attributes and petrophysical attributes) of subterranean points in the reservoir region 101 from the memory 1106. The memory 1106 may obtain characteristics of subterranean points from a variety of geological data sources and geophysical data sources. In one example, using sensors and the geological analyzer 670, the processor 650 may collect remote sensing geophysical surveys, such as seismic surveys, gravity surveys, and active and passive source resistivity surveys. In other examples, the processor 650 may collect data such as well logs, core data, production data, acquired in wells in the reservoir region 101 to determine geological and petrophysical characteristics of the subterranean points.

In some embodiments, the processor 650 may receive petrophysical characteristics such as pore radius and permeability and/or geological attributes of the subterranean points from the server to which measurements of geological and/or petrophysical attributes are submitted from the logging unit 630, the petrophysical analyzer 640, and the like. The processor 650 may analyze the characteristics of the subterranean points (including the plurality of sample points) that the logging unit 630, the memory 1106, and the server obtain.

In accordance with one or more embodiments, geological, physical, and petrophysical properties of the subterranean points may be estimated from other attributes of the subterranean points, as will be discussed later in detail.

The processor 650 may be programmed to assign a geological category and a petrophysical category for each sample point within the plurality of sample points based, at least in part, on the obtained characteristics.

In accordance with one or more embodiments, the processor 650 may be programmed to assign a hybrid category to each sample point within the plurality of sample points based, at least in part, on the geological category and the petrophysical category. More specifically, the processor 650 will apply a classification of hybrid category, for example, as set forth in a table in FIG. 5C.

In one or more embodiments, the processor 650 is programmed to generate the virtual model of subterranean region based, at least in part, on the hybrid category assigned to each sample point within the plurality of sample points. The virtual model of subterranean region is a three-dimensional representation of the distribution of hybrid categories. The virtual model of subterranean region may take a form of a map illustrating a three-dimensional distribution of the hybrid category in the reservoir region 101.

The generated virtual model of subterranean region facilitates oil and gas exploration as follows.

In general, prior to the commencement of drilling, a wellbore plan is usually generated. The wellbore plan may include a starting surface location of the production wells 102, or a subsurface location within an existing wellbore, from which a wellbore may be drilled. Further, the wellbore plan may include a terminal location that may intersect with targeted hydrocarbon bearing formation and a planned wellbore path from the starting location to the terminal location. Once resource-rich rocks in the reservoir region 101 are located, an optimal wellbore plan may become clear. To grasp rock characteristics of a subterranean region, pertinent information is collected by testing and surveys, including examinations performed by a logging unit.

As shown in FIG. 6B, a wellbore 601 following a wellbore trajectory 602 may be drilled by a drill bit 603 attached by a drillstring 604 to a drill rig 605 located on the surface 606 of the earth. The drill rig 605 may include framework, such as a derrick 607 to hold drilling machinery. A top drive 608 sits at the top of the derrick 607 and provides clockwise torque via the drive shaft 609 to the drillstring 604 in order to drill the wellbore 601. The drillstring 604 may comprise a plurality of sections of drillpipe attached at the uphole end to the drive shaft 609 and downhole to a bottomhole assembly (“BHA”) 610. The BHA may be composed of a plurality of sections of heavier drillpipe and one or more measurement-while-drilling (“MWD”) tools configured to measure drilling parameters, such as torque, weight-on-bit, drilling direction, temperature, etc., and one or more logging-while-drilling (“LWD”) tools configured to measure parameters of the rock surrounding the wellbore 601, such as electrical resistivity, density, sonic propagation velocities, gamma-ray emission, etc.

The wellbore 601 may traverse a plurality of overburden 611 layers and one or more cap-rock 612 layers to a hydrocarbon reservoir 104 within the subterranean region, and specifically to a drilling target 613 within the hydrocarbon reservoir 104. The wellbore trajectory 602 may be a curved or a straight trajectory. All or part of the wellbore trajectory 602 may be vertical, and some wellbore trajectory 602 may be deviated or have horizontal sections. One or more portions of the wellbore 601 may be cased with casing 615 in accordance with the wellbore plan.

To start drilling, or “spudding in” the well, the hoisting system lowers the drillstring 604 suspended from the derrick 607 towards the planned surface location of the wellbore. An engine, such as a diesel engine, may be used to supply power to the top drive 608 to rotate the drillstring 604. The weight of the drillstring 604 combined with the rotational motion enables the drill bit 603 to bore the wellbore.

The near-surface is typically made up of loose or soft sediment or rock, so large diameter casing 615, e.g., “base pipe” or “conductor casing,” is often put in place while drilling to stabilize and isolate the wellbore. At the top of the base pipe is the wellhead, which serves to provide pressure control through a series of spools, valves, or adapters. Once near-surface drilling has begun, water or drill fluid may be used to force the base pipe into place using a pumping system until the wellhead is situated just above the surface 606 of the earth.

Still at FIG. 6B, drilling may continue without any casing 615 once deeper, or more compact rock is reached. While drilling, a drilling mud system 614 may pump drilling mud from a mud tank on the surface 606 through the drill pipe. Drilling mud serves various purposes, including pressure equalization, removal of rock cuttings, and drill bit cooling and lubrication.

At planned depth intervals, drilling may be paused and the drillstring 604 withdrawn from the wellbore. Sections of casing 615 may be connected and inserted and cemented into the wellbore. Casing string may be cemented in place by pumping cement and mud, separated by a “cementing plug,” from the surface 606 through the drill pipe. The cementing plug and drilling mud force the cement through the drill pipe and into the annular space between the casing and the wellbore wall. Once the cement cures, drilling may recommence. The drilling process is often performed in several stages. Therefore, the drilling and casing cycle may be repeated more than once, depending on the depth of the wellbore and the pressure on the wellbore walls from surrounding rock.

Due to the high pressures experienced by deep wellbores, a blowout preventer (BOP) may be installed at the wellhead to protect the rig and environment from unplanned oil or gas releases. As the wellbore becomes deeper, both successively smaller drill bits and casing string may be used. Drilling deviated or horizontal wellbores may require specialized drill bits or drill assemblies.

The drilling unit 600 may be disposed at and communicate with other systems in the well environment. The drilling unit 600 may control at least a portion of a drilling operation by providing controls to various components of the drilling operation. In one or more embodiments, the system of creating a virtual model of subterranean region may receive data from one or more sensors arranged to measure controllable parameters of the drilling operation. As a non-limiting example, sensors may be arranged to measure weight-on-bit, drill rotational speed (RPM), flow rate of the mud pumps (GPM), and rate of penetration of the drilling operation (ROP). Each sensor may be positioned or configured to measure a desired physical stimulus. Drilling may be considered complete when a drilling target 613 is reached, or the presence of hydrocarbons is established.

The following paragraphs explain how a category (a geological, a petrophysical, or a hybrid) of a sample point within the plurality of sample points may be determined when relevant characteristics of the sample point are not available.

Machine learning (ML), broadly defined, is the extraction of patterns and insights from data. The phrases “artificial intelligence,” “machine learning,” “deep learning,” and “pattern recognition” are often convoluted, interchanged, and used synonymously. This ambiguity arises because the field of “extracting patterns and insights from data” was developed simultaneously and disjointedly among a number of classical arts like mathematics, statistics, and computer science. For consistency, the term machine learning, or machine learned, will be adopted herein. However, one skilled in the art will recognize that the concepts and methods detailed hereafter are not limited by this choice of nomenclature.

In some embodiments, the ML model may be a recurrent convolutional neural network (RCNN), such as the Pixel convolutional neural network (PixelCNN). An RCNN may be more readily understood as a specialized neural network (NN) and, from there, as a specialized convolutional neural network (CNN). Thus, a cursory introduction to an NN and a CNN are provided herein. However, note that many variations of an NN and CNN exist. Therefore, one of ordinary skill in the art will recognize that any variation of an NN or CNN (or any other ML model) may be employed without departing from the scope of this disclosure. Further, it is emphasized that the following discussions of an NN and CNN are basic summaries and should not be considered limiting.

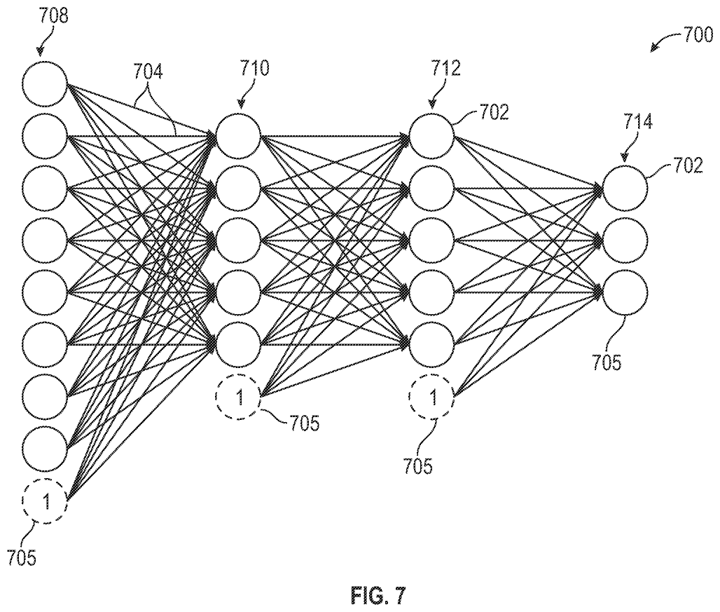

A diagram of an NN is shown in FIG. 7. At a high level, an NN 700 may be graphically depicted as being composed of nodes 702 and edges 704. The nodes 702 may be grouped to form layers 705. FIG. 7 displays four layers 708, 710, 712, 714 of nodes 702 where the nodes 702 are grouped into columns. However, each group need not be as shown in FIG. 7. The edges 704 connect the nodes 702 to other nodes 702. Edges 704 may connect, or not connect, to any node(s) 702 regardless of which layer 705 the node(s) 702 is in. That is, the nodes 702 may be sparsely and residually connected. For example, in a recurrent neural network (RNN), nodes 702 in the output layer 714 may be connected by edges 704 to nodes 702 in the input layer 708 (though not shown in FIG. 1).

An NN 700 will have at least two layers 705, where the first layer 708 is the “input layer” and the last layer 714 is the “output layer.” Any intermediate layer 710, 712 is usually described as a “hidden layer.” An NN 700 may have zero or more hidden layers 710, 712. An NN 700 with at least one hidden layer 710, 712 may be described as a “deep” neural network or “deep learning method.” In general, an NN 700 may have more than one node 702 in the output layer 714. In these cases, the neural network 700 may be referred to as a “multi-target” or “multi-output” network.

Nodes 702 and edges 704 carry associations. Namely, every edge 704 is associated with a numerical value. The edge numerical values, or even the edges 704 themselves, are often referred to as “weights” or “parameters.” While training an NN 700, a process that will be described below, numerical values are assigned to each edge 704. Additionally, every node 702 is associated with a numerical value and may also be associated with an activation function. Activation functions are not limited to any functional class, but traditionally follow the form:

$$A = f ( {\sum }_{i ∈ ( incoming )} [ {( node value )}_{i} {( edge value )}_{i} ] ) , \qquad Equation ( 1 )$$

where i is an index that spans the set of “incoming” nodes 702 and edges 704 and f is a user-defined function. Incoming nodes 702 are those that, when viewed as a graph (as in FIG. 7), have directed arrows that point to the node 702 where the numerical value is being computed. Some functions ƒ may include the linear function ƒ(x)=x, sigmoid function

$$f ( x ) = \frac{1}{1 + {e}^{- x}} ,$$

and rectified linear unit function ƒ(x)=max (0, x), however, many additional functions are commonly employed. Every node 702 in an NN 700 may have a different associated activation function. Often, as a shorthand, activation functions are described by the function ƒ by which it is composed. That is, an activation function composed of a linear function ƒ may simply be referred to as a linear activation function without undue ambiguity.

When the NN 700 receives an input, the input is propagated through the network according to the activation functions and incoming node values and edge values to compute a value for each node 702. That is, the numerical value for each node 702 may change for each received input while the edge values remain unchanged. Occasionally, nodes 702 are assigned fixed numerical values, such as the value of 1. These fixed nodes 706 are not affected by the input or altered according to edge values and activation functions. Fixed nodes 706 are often referred to as “biases” or “bias nodes” as displayed in FIG. 7 with a dashed circle.

In some implementations, the NN 700 may contain specialized layers 705, such as a normalization layer, pooling layer, or additional connection procedures, like concatenation. One skilled in the art will appreciate that these alterations do not exceed the scope of this disclosure.

The number of layers in an NN 700, choice of activation functions, inclusion of batch normalization layers, and regularization strength, among others, may be described as “hyperparameters” that are associated to the ML model. It is noted that in the context of ML, the regularization of a ML model refers to a penalty applied to the loss function of the ML model. The selection of hyperparameters associated to a ML model is commonly referred to as selecting the ML model “architecture.”

Once a ML model, such as an NN 700, and associated hyperparameters have been selected, the ML model may be trained. To do so, M training pairs may be provided to the NN 700, where M is an integer greater than or equal to one. The variable m maintains a count of the M training pairs. As such, m is an integer between 1 and M inclusive of 1 and M where m is the current training pair of interest. For example, if M=2, the two training pairs include a first training pair and a second training pair each of which may be generically denoted an mth training pair. In general, each of the M training pairs includes an input and an associated target output. Each associated target output represents the “ground truth,” or the otherwise desired output upon processing the input. During training, the NN 700 processes at least one input from an mth training pair in the form of an mth training geological data patch to produce at least one output. Each NN output is then compared to the associated target output from the mth training pair in the form of an mth training feature image patch.

Returning to the NN 700 in FIG. 7, the NN 700 may be trained by first assigning initial values to the edges 704. These values may be assigned randomly, according to a prescribed distribution, manually, or by some other assignment mechanism. Once edge values have been initialized, the NN 700 may act as a function such that it may receive an input from an mth training pair and produce an output. At least one input is propagated through the neural network 700 to produce an output. The M training pairs will be discussed in more detail below.

The comparison of the NN output to the associated target output from the mth training pair is typically performed by a “loss function.” Other names for this comparison function include an “error function,” “misfit function,” and “cost function.” Many types of loss functions are available, such as the log-likelihood function. However, the general characteristic of a loss function is that the loss function provides a numerical evaluation of the similarity between the NN output and the associated target output from the mth training pair. The loss function may also be constructed to impose additional constraints on the values assumed by the edges 704. For example, a penalty term, which may be physics-based, or a regularization term may be added. Generally, the goal of a training procedure is to alter the edge values to promote similarity between the NN output and associated target output for most, if not all, of the M training pairs. Thus, the loss function is used to guide changes made to the edge values. This process is typically referred to as “backpropagation.”

While a full review of the backpropagation process exceeds the scope of this disclosure, a brief summary is provided. Backpropagation consists of computing the gradient of the loss function over the edge values. The gradient indicates the direction of change in the edge values that results in the greatest change to the loss function. Because the gradient is local to the current edge values, the edge values are typically updated by a “step” in the direction indicated by the gradient. The step size is often referred to as the “learning rate” and need not remain fixed during the training process. Additionally, the step size and direction may be informed by previous edge values or previously computed gradients. Such methods for determining the step direction are usually referred to as “momentum” based methods.

Once the edge values of the NN 700 have been updated through the backpropagation process, the NN 700 will likely produce different outputs than it did previously. Thus, the procedure of propagating at least one input from an mth training pair through the NN 700, comparing the NN output with the associated target output from the mth training pair with a loss function, computing the gradient of the loss function with respect to the edge values, and updating the edge values with a step guided by the gradient is repeated until a termination criterion is reached. Common termination criteria include, but are not limited to, reaching a fixed number of edge updates (otherwise known as an iteration counter), reaching a diminishing learning rate, noting no appreciable change in the loss function between iterations, or reaching a specified performance metric as evaluated on the m training pairs or separate hold-out training pairs (often denoted “validation data”). Once the termination criterion is satisfied, the edge values are no longer altered and the neural network 700 is said to be “trained.”

Turning to a CNN, a CNN is similar to an NN 700 in that it can technically be graphically represented by a series of edges 704 and nodes 702 grouped to form layers 705. However, it is more informative to view a CNN as structural groupings of weights. Here, the term “structural” indicates that the weights within a group have a relationship, often a spatial relationship. CNNs are widely applied when the input also has a relationship. For example, the pixels of a seismic image have a spatial relationship where the value associated to each pixel is spatially dependent on the value of other pixels of the seismic image. Consequently, a CNN is an intuitive choice for processing geological data that includes a seismic image and may include other spatially dependent data.

A structural grouping of weights is herein referred to as a “filter” or “convolution kernel.” The number of weights in a filter is typically much less than the number of inputs, where, now, each input may refer to a pixel in an image. For example, a filter may take the form of a square matrix, such as a 3×3 or 7×7 matrix. In a CNN, each filter can be thought of as “sliding” over, or convolving with, all or a portion of the inputs to form an intermediate output or intermediate representation of the inputs which possess a relationship. The portion of the inputs convolved with the filter may be referred to as a “receptive field.” Like the NN 700, the intermediate outputs are often further processed with an activation function. Many filters of different sizes may be applied to the inputs to form many intermediate representations. Additional filters may be formed to operate on the intermediate representations creating more intermediate representations. This process may be referred to as a “convolutional layer” within the CNN. Multiple convolutional layers may exist within a CNN as prescribed by a user.

There is a “final” group of intermediate representations, wherein no filters act on these intermediate representations. In some instances, the relationship of the final intermediate representations is ablated, which is a process known as “flattening.” The flattened representation may be passed to an NN 700 to produce a final output. Note that, in this context, the NN 700 is considered part of the CNN.

Like an NN 700, a CNN is trained. The filter weights and the edge values of the internal NN 700, if present, are initialized and then determined using the M training pairs and backpropagation as previously described.

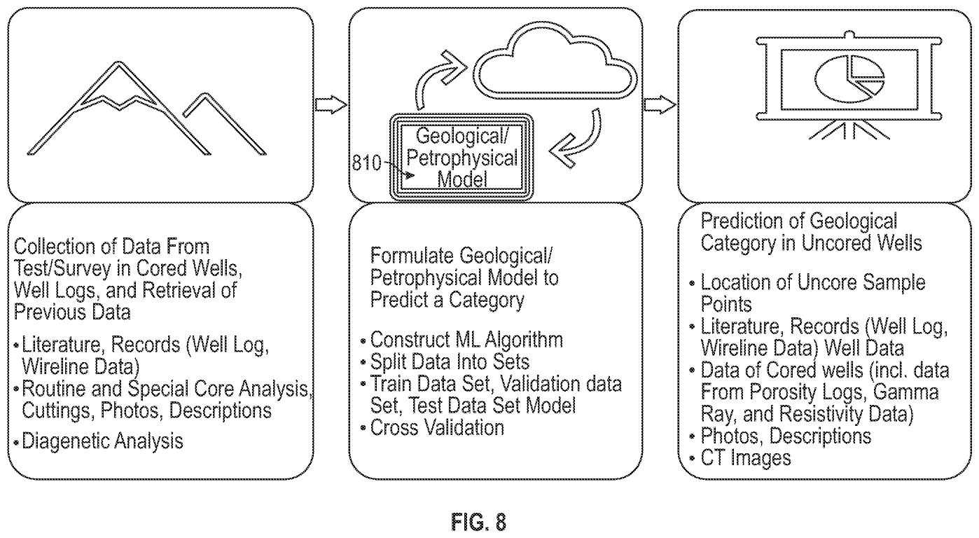

Turning to FIG. 8, FIG. 8 shows a schematic diagram describing the system of generating a virtual subterranean region in accordance with one or more embodiments.

Modified Dunham Classification is a time-saving process compared with Traditional Dunham Classification and does not need a lot of characteristics to determine a geological category. Even so, there may be instances where it is not possible to determine a geological category even under Modified Dunham Classification. In such situations, the processor 650 generates a geological/petrophysical model 810 based, at least in part, on available characteristics in order to predict a geological/petrophysical category of a sample point.

The first column of FIG. 8 concerns the collection of characteristics of subterranean points. As explained above, in certain implementations, characteristics of subterranean points within the reservoir region 101 may be obtained from a variety of geological and geophysical sources. The characteristics may be collected from previously generated well logs, wireline data, or production records as acquired in nearby wells penetrating the reservoir region 101.

In certain implementations, the processor 650 may retrieve the characteristics of the plurality of sample points obtained by the logging unit 630 in the wellbore 601 of the production well 102. The rock density, the seismic velocity, and the resistivity of rock samples may be measured along the wellbore 601.

In some examples, data from remote sensing geophysical surveys, such as seismic surveys, gravity surveys, and active and passive source resistivity surveys are obtained. In yet other implementations, the processor 650 receives the characteristics of the subterranean points from the server which stores data from various sources. The server may store: petrophysical attributes and geological attributes of rock samples generated by the petrophysical analyzer 640 (including routine and special core analysis and description of rock samples), the geological analyzer 670, and by other examination tools (including cutting and photography) collected along the wellbore 601; and the analysis of petrophysical attributes and geological attributes of the subterranean points (including the plurality of sample points) generated by the processor 650.

Moving on to the second column of FIG. 8, the processor 650 may formulate a geological/petrophysical model 810 using ML networks (including those to perform a classification analysis) to predict a geological/petrophysical category of each of the plurality of sample points in accordance with one or more embodiments.

Prior to and during the formulation of the geological/petrophysical model 810, the processor 650 may collect various data, for example, a well log covering the plurality of subterranean points within the wellbore 601, generated at the logging unit 630. The collected data may include previously assigned geological categories (under Modified Dunham Classification in FIG. 5A), petrophysical categories, geological attributes, permeability data of subterranean points and may further include data received from the server and the memory 1106. In some embodiments, the processor 650 divides the collected data into a few sets and trains the geological/petrophysical model 810 using the well log of the production well 102 and the geological/petrophysical categories of the plurality of sample points in cored intervals with available porosity logs and other log data (for example, gamma ray and resistivity data), to obtain the geological/petrophysical model 810 and to predict geological/categories in un-cored intervals, operating a machine learning (ML) network. Collected data in other sets are used for validating and/or testing the geological/petrophysical model 810 in accordance with one or more embodiments.

In some implementations, geological attributes of the subterranean points are used as inputs to train or test the prediction of a geological category (output) by the geological/petrophysical model 810. In such implementations, the processor 650 may validate the geological/petrophysical model 810 by cross-validating among the data sets.

In one or more embodiments, the processor 650 may perform the classification analysis at least in part, at the server, using the collected data. In other embodiments, the classification analysis is performed by the processor 650.

In example implementations of the system, the processor 650 may generate the geological/petrophysical model 810 by generating a NN based on Bayes's theorem.

As one example of the system, the processor 650 may construct the geological/petrophysical model 810 on support vector machine, and a subset of training data may be used in a decision function. As other examples, the classification analysis may run K-Nearest Neighbor to the collected data.

At the last column of FIG. 8, when the geological/petrophysical model 810 is formulated and validated, and tested in some instances, the processor 650 determines a geological/petrophysical category for each analysis point of the plurality of analysis points in the wellbore 601 using the collected data, including data from the well log covering the plurality of analysis points.

In certain implementations, the processor 650 returns a geological/petrophysical category of an analysis point to which there is no core sample. In such circumstances, the collected data may not include petrophysical attributes and certain geological attributes of the analysis point.

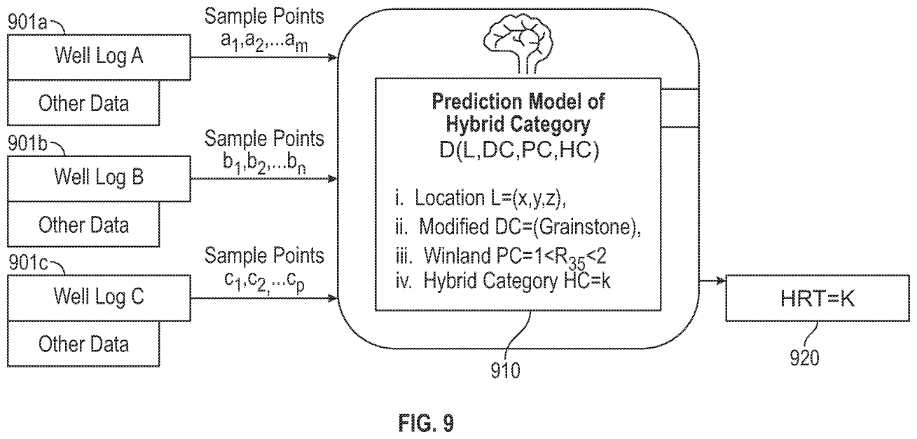

Turning to FIG. 9, FIG. 9 shows one example of determination of a hybrid category by the processor 650, using machine learning.

In some embodiments, the processor 650 may formulate a prediction model 910 of a hybrid category on NN. For example, the prediction model may be built as one type of classification analysis. The architecture may be generated by determining an activation function, a number of hidden layers, hyperparameters, to predict a hybrid category of a plurality of prediction points (c₁, c₂, . . . cₚ) from a well log 901c containing a plurality of prediction points (c₁, c₂, . . . cₚ) in a second wellbore. In such embodiments, the second wellbore may be the wellbore 601 or a different wellbore.

In one or more embodiments, the processor 650 may collect various data including the well log 901a, 901b, covering the plurality of sample points (a₁, a₂, . . . aₘ), (b₁, b₂, . . . bₙ) within the wellbore of the production well 102, a well log 901c covering the plurality of prediction points (c₁, c₂, . . . cₚ) within a second wellbore, which may be generated at a logging unit 630 in accordance with one or more embodiments. The second wellbore may be the wellbore 601, and a logging unit may be the logging unit 630 in some examples.

In some embodiments, the processor 650 assigns a geological category (GC) and a petrophysical category (PC) to each of the plurality of sample points (a₁, a₂, . . . aₘ), (b₁, b₂, . . . bₙ) based on the characteristics of the each of the plurality of sample points (a₁, a₂, . . . aₘ), (b₁, b₂, . . . bₙ) and thereafter, assigns a hybrid category (HC) to each of the plurality of sample points (a₁, a₂, . . . aₘ), (b₁, b₂, . . . bₙ).

As to any of the plurality of sample points (a₁, a₂, . . . aₘ), (b₁, b₂, . . . bₙ) and the plurality of prediction points (c₁, c₂, . . . cₚ) that lack either a core sample or a well log, or both of them, the processor 650 may predict its geological/petrophysical category using the geological/petrophysical model 810. For example, the permeability and/or a petrophysical category of Winland rock typing may be predicted for sample points (a₁, a₂, . . . aₘ), (b₁, b₂, . . . bₙ) using the geological/petrophysical model 810 to be used in the determination of a hybrid category.

In some embodiments, the processor 650 divides the collected data into a few sets and train the prediction model 910 using the well log 901a and the hybrid category (HC) of each sample point in the plurality of sample points (a₁, a₂, . . . aₘ), (b₁, b₂, . . . bₙ) operating a machine learning (ML) network. Data in other sets are used for validating or testing the prediction model 910 in accordance with several embodiments.

In one or more embodiments, the processor 650 may generate the prediction model 910 as a function of a location of a point (x, y, z) within the reservoir region 101, by executing an algorithm based on Bayes's theorem.

In other embodiments, the system may construct the prediction model 910 by the establishment of an architecture on support vector machine.

Still with FIG. 9, when the prediction model 910 is formulated and validated, and tested in some instances, the processor 650 predicts a hybrid category for each of the plurality of prediction points (c₁, c₂, . . . cₚ) in the second wellbore based on their respective geological category and petrophysical category, which are assigned by the processor 650 after analyzing the collected data including the well log 901c covering the plurality of prediction points (c₁, c₂, . . . cₚ).

Upon collection of the characteristics of each of the plurality of prediction points (c₁, c₂, . . . cₚ) from various resources such as the logging unit 630, the memory 1106, and the server, the processor 650 analyzes the characteristics and generates or updates the prediction of the hybrid category.

The virtual model of a subterranean region may be produced as a digital representation of three-dimensional distribution of the hybrid category assigned to the subterranean points, including the plurality of sample points (a₁, a₂, . . . aₘ), (b₁, b₂, . . . bₙ), and the plurality of prediction points (c₁, c₂, . . . cₚ). In some embodiments, when the processor 650 determines a hybrid category of any of the plurality of sample points (a₁, a₂, . . . aₘ), (b₁, b₂, . . . bₙ), a three-dimensional graph indicating the hybrid category of each sample point is generated by the processor 650 and displayed by the display.

In further embodiments, when the processor 650 predicts a hybrid category of any of the plurality of prediction points (c₁, c₂, . . . cₚ), the virtual model of subterranean region is generated as a graph of the spatial distribution of the predicted hybrid category for each prediction point of the plurality of prediction points (c₁, c₂, . . . cₚ) and displayed by the display.

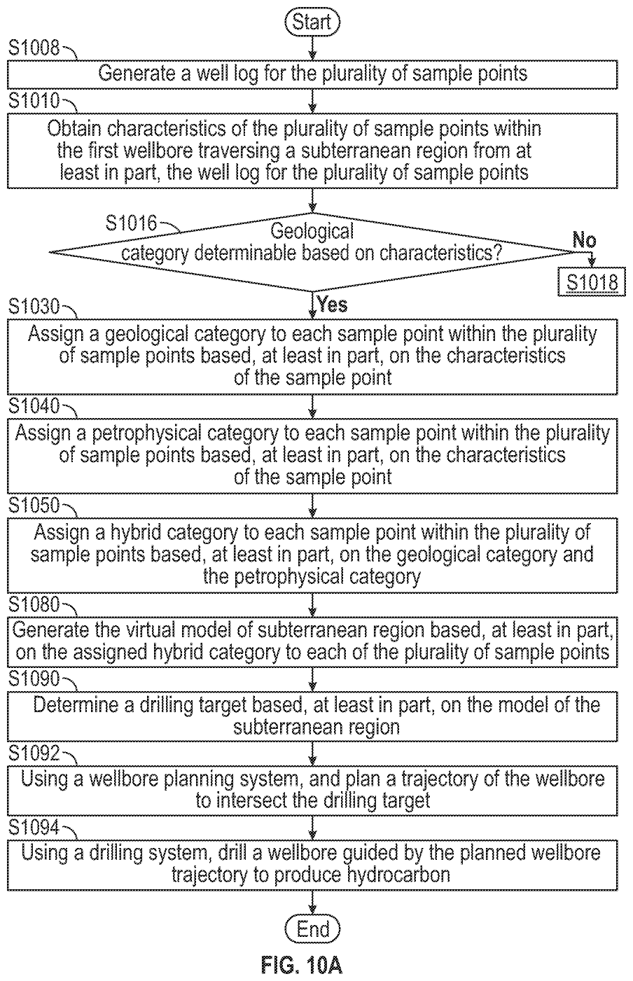

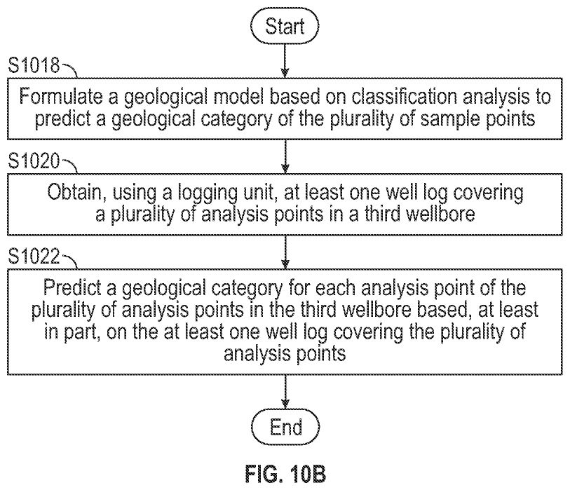

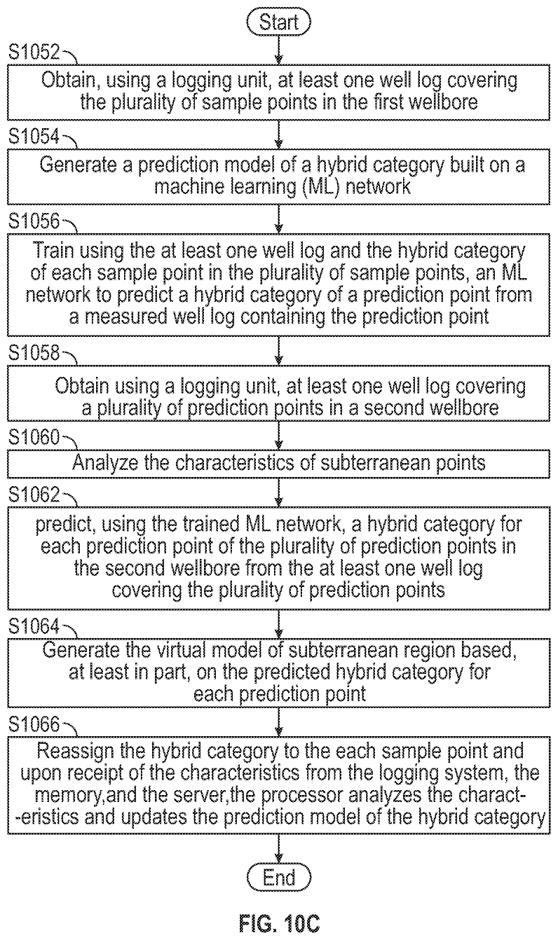

FIGS. 10A to 10C show flowcharts of a method for of generating a virtual model of subterranean region in accordance with one or more embodiments. One or more of the individual steps shown in FIGS. 10A to 10C may be omitted, repeated, and/or performed in a different order than the order shown in FIGS. 10A to 10C. Accordingly, the scope of the disclosure should not be limited by the specific arrangement as depicted.

Referring to FIG. 10A, at S1000, the system, via the processor 650, determines whether core samples will be obtained within the first wellbore 601 and how many in one or more embodiments. If a core sample will be obtained by the core collection unit 620, the step proceeds to S1002. If no core sample will be obtained, the step proceeds to S1008.

At S1002, the core collection unit 620 obtains a core sample at some of the plurality of sample points in the well 102.

Using the core sample, at S1004, the geological analyzer 670 performs a geological examination, such as a visual inspection and/or a microscopic examination. As a result of the examination, geological characteristics are obtained and submitted to the server or the memory 1106.

Further, at S1006, the processor 650 directs the petrophysical analyzer 640 to perform petrophysical analysis of the core sample in accordance with one or more embodiments. As a result of the examination, petrophysical characteristics are obtained and submitted to the server in some embodiments.

At S1008, the step proceeds to generating a well log for the plurality of sample points by the logging unit 630. The logging unit 630, which may include logging tools, measures and collects characteristics of the plurality of sample points within the wellbore 601 in accordance with one or more embodiments.

At S1010, the system, via the processor 650, obtains characteristics of the plurality of sample points within the first wellbore 601 traversing a subterranean region from at least in part, the well log for the plurality of sample points in accordance with one or more embodiments.

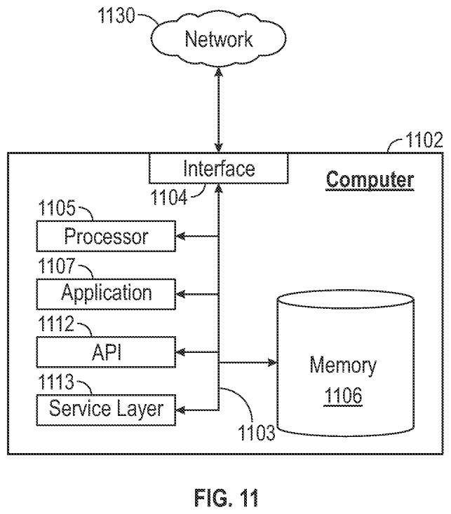

Optionally, the system, via the processor 650, obtains characteristics of subterranean points, including the plurality of sample points, using various resources, in accordance with one or more embodiments. In some implementations, the processor 650 retrieves geological and petrophysical characteristics of the subterranean points in the reservoir region 101 from the memory 1106 (see FIG. 11).

The memory 1106 may obtain characteristics of the subterranean points within the reservoir region 101 from a variety of sources. As one example, data from remote sensing geophysical surveys, such as seismic surveys, gravity surveys, and active and passive source resistivity surveys, may be obtained. In addition, collected data such as well logs and core data, which may be acquired in other wells penetrating the reservoir region 101, may be used to determine geological and petrophysical characteristics.

In some embodiments, the processor 650 may receive petrophysical characteristics such as pore radius and permeability and/or geological attributes of the subterranean points from the server. In one example, the porosity, the permeability, the density, the seismic velocity, and the resistivity of rock samples may be measured on the core sample or on spots.

In accordance with one or more embodiments, remote sensing geophysical surveys and physical and petrophysical attributes determined from well logs may be combined to estimate physical and petrophysical characteristics.

The processor 650 analyzes the characteristics of the subterranean points (including the plurality of sample points) that the logging unit 630, the memory 1106, and the server obtain.

At S1016, the processor 650 determines whether a geological category of each of the plurality of sample points is determinable based on collected characteristics. If the geological category is determinable, the step proceeds to S1030. If the geological category is not determinable, the step proceeds to S1018.

In FIG. 10B, at S1018, the processor 650 formulates a geological model, for example, based on classification analysis that relates a determined geological category of each sample point of the plurality of sample points to the characteristics of each sample point of the plurality of sample points.

At S1020, the processor 650 obtains, using a logging unit, at least one well log covering a plurality of analysis points in a third wellbore in accordance with one or more embodiments.

At S1022, the processor 650 predicts a geological category for each analysis point of the plurality of analysis points in the third wellbore based, at least in part, on the at least one well log covering the plurality of analysis points.

Referring back to FIG. 10A, at S1030, the processor 650 assigns a geological category to each sample point within the plurality of sample points based, at least in part, on the characteristics of the sample point.

At S1040, the processor 650 assigns a petrophysical category to each sample point within the plurality of sample points based, at least in part, on the characteristics of the sample point.

At S1042, the processor 650 determines whether a geological category and a petrophysical category are assigned to each sample point within the plurality of sample points based, at least in part, on the characteristics of the sample point in accordance with one or more embodiments. If both the geological category and the petrophysical category are assigned, the step proceeds to S1050. If either the geological category or the petrophysical category is not assigned, the step proceeds to S1052.

At S1050, the processor 650 assigns a hybrid category to each sample point within the plurality of sample points based, at least in part on the geological category and the petrophysical category.

Turning to FIG. 10C, at S1052, when the petrophysical category or the geological category is not assigned to each sample point within the plurality of sample points, the processor 650 obtains, at least one well log 901a, 901b, covering the plurality of sample points (a₁, a₂, . . . aₘ), (b₁, b₂, . . . bₙ) in the first wellbore 601.

At S1054, the processor 650 generates a prediction model 910 of a hybrid category built on a machine learning network to predict a hybrid category of each of a plurality of prediction points.

At S1056, the processor 650 trains the prediction model 910 on ML network, using the at least one well log 901a, 901b and the hybrid category of each sample point in the plurality of sample points (a₁, a₂, . . . aₘ), (b₁, b₂, . . . bₙ) for example, based on classification analysis.

At S1058, the processor 650 obtains, using a logging unit, at least one well log 901c covering a plurality of prediction points (c₁, c₂, . . . cₚ) in a second wellbore in accordance with one or more embodiments.

The first wellbore 601 may comprise the second wellbore or the third wellbore in some embodiments.

At S1060, the processor 650 analyzes the characteristics of subterranean points. The characteristics may be found in well logs, petrophysical property measurements, seismic data, and/or other types of data.

At S1062, the processor 650 predicts, using the trained prediction model built on machine learning architecture, a hybrid category for each prediction point of the plurality of prediction points (c₁, c₂, . . . cₚ) from the at least one well log 901c in some embodiments.

At S1064, the processor 650 generates the virtual model of subterranean region based, at least in part, on the predicted hybrid category for each prediction point of the plurality of prediction points (c1, c2, . . . cp) in accordance with one or more embodiments.

At S1066, the processor 650 may be programmed to reassign a hybrid category to each sample point within the plurality of sample points. Upon receipt of the characteristics from the logging unit 630, the memory 1106, and the server, the processor 650 analyzes the characteristics and updates the prediction model 910 of the hybrid category.

Referring back to FIG. 10A, at S1080, the processor 650 generates the virtual model of subterranean region based, at least in part, on the hybrid category assigned to each sample point in the plurality of sample points.

Moving on, at S1090, the processor 650 determines a drilling target 613 based, at least in part, on the model of the subterranean region in accordance with one or more embodiments.

At S1092, the processor 650 directs the wellbore planning unit 660 to determine a drilling target 613 based, at least in part, on the virtual model of subterranean region, and plan a trajectory 602 of the wellbore 601 to intersect the drilling target 613 in accordance with one or more embodiments. In some embodiments, the processor 650 may include the wellbore planning unit 660. In such embodiments, the planned wellbore trajectory 602 guides the drilling unit 600 in drilling the wellbore 601.

At S1094, the processor 650 directs drilling, using the drilling unit 600, the wellbore 601 guided by the planned wellbore trajectory 602 to produce hydrocarbon.

FIG. 11 depicts a block diagram of a computer system used to provide computational functionalities associated with described algorithms, methods, functions, processes, flows, and procedures as described in this disclosure, according to one or more embodiments. The illustrated computer (1102) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (1102) may include a computer that includes an input device, such as a keypad, keyboard, touch screen, or other device that can accept user information, and an output device that conveys information associated with the operation of the computer (1102), including digital data, visual, or audio information (or a combination of information), or a GUI.

The computer (1102) can serve in a role as a client, network component, a server, a database or other persistency, or any other component (or a combination of roles) of a computer system for performing the subject matter described in the instant disclosure. The illustrated computer (1102) is communicably coupled with a network (1130). In some implementations, one or more components of the computer (1102) may be configured to operate within environments, including cloud-computing-based, local, global, or other environment (or a combination of environments).

At a high level, the computer (1102) is an electronic computing device operable to receive, transmit, process, store, or manage data and information associated with the described subject matter. According to some implementations, the computer (1102) may also include or be communicably coupled with an application server, e-mail server, web server, caching server, streaming data server, business intelligence (BI) server, or other server (or a combination of servers).

The computer (1102) can receive requests over network (1130) from a client application (for example, executing on another computer (1102) and responding to the received requests by processing the said requests in an appropriate software application. In addition, requests may also be sent to the computer (1102) from internal users (for example, from a command console or by other appropriate access method), external or third-parties, other automated applications, as well as any other appropriate entities, individuals, systems, or computers.

Each of the components of the computer (1102) can communicate using a system bus (1103). In some implementations, any or all of the components of the computer (1102), both hardware or software (or a combination of hardware and software), may interface with each other or the interface (1104) (or a combination of both) over the system bus (1103) using an application programming interface (API) (1112) or a service layer (1113) (or a combination of the API (1112) and service layer (1113). The API (1112) may include specifications for routines, data structures, and object classes. The API (1112) may be either computer-language independent or dependent and refer to a complete interface, a single function, or even a set of APIs. The service layer (1113) provides software services to the computer (1102) or other components (whether or not illustrated) that are communicably coupled to the computer (1102). The functionality of the computer (1102) may be accessible for all service consumers using this service layer. Software services, such as those provided by the service layer (1113), provide reusable, defined business functionalities through a defined interface. For example, the interface may be software written in JAVA, C++, or other suitable language providing data in extensible markup language (XML) format or another suitable format. While illustrated as an integrated component of the computer (1102), alternative implementations may illustrate the API (1112) or the service layer (1113) as stand-alone components in relation to other components of the computer (1102) or other components (whether or not illustrated) that are communicably coupled to the computer (1102). Moreover, any or all parts of the API (1112) or the service layer (1113) may be implemented as child or sub-modules of another software module, enterprise application, or hardware module without departing from the scope of this disclosure.

The computer (1102) includes an interface (1104). Although illustrated as a single interface (1104) in FIG. 11, two or more interfaces (1104) may be used according to particular needs, desires, or particular implementations of the computer (1102). The interface (1104) is used by the computer (1102) for communicating with other systems in a distributed environment that are connected to the network (1130). Generally, the interface (1104) includes logic encoded in software or hardware (or a combination of software and hardware) and operable to communicate with the network (1130). More specifically, the interface (1104) may include software supporting one or more communication protocols associated with communications such that the network (1130) or interface's hardware is operable to communicate physical signals within and outside of the illustrated computer (1102).

The computer (1102) includes at least one computer processor (1105). Although illustrated as a single computer processor (1105) in FIG. 11, two or more processors may be used according to particular needs, desires, or particular implementations of the computer (1102). Generally, the computer processor (1105) executes instructions and manipulates data to perform the operations of the computer (1102) and any algorithms, methods, functions, processes, flows, and procedures as described in the instant disclosure.

The computer (1102) also includes a memory (1106) that holds data for the computer (1102) or other components (or a combination of both) that can be connected to the network (1130). For example, memory (1106) can be a database storing data consistent with this disclosure. Although illustrated as a single memory (1106) in FIG. 11, two or more memories may be used according to particular needs, desires, or particular implementations of the computer (1102) and the described functionality. While memory (1106) is illustrated as an integral component of the computer (1102), in alternative implementations, memory (1106) can be external to the computer (1102).

The application (1107) is an algorithmic software engine providing functionality according to particular needs, desires, or particular implementations of the computer (1102), particularly with respect to functionality described in this disclosure. For example, application (1107) can serve as one or more components, modules, applications, etc. Further, although illustrated as a single application (1107), the application (1107) may be implemented as multiple applications (1107) on the computer (1102). In addition, although illustrated as integral to the computer (1102), in alternative implementations, the application (1107) can be external to the computer (1102).

There may be any number of computers (1102) associated with, or external to, a computer system containing computer (1102), wherein each computer (1102) communicates over network (1130). Further, the term “client,” “user,” and other appropriate terminology may be used interchangeably as appropriate without departing from the scope of this disclosure. Moreover, this disclosure contemplates that many users may use one computer (1102), or that one user may use multiple computers (1102).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method of generating a virtual model of a subterranean region, comprising:
obtaining characteristics of a plurality of sample points within a first wellbore traversing the subterranean region;
for each sample point, within the plurality of sample points:
assigning a geological category based, at least in part, on the characteristics of the each sample point, and
assigning a petrophysical category based, at least in part, on the characteristics of the each sample point,
assigning a hybrid category based, at least in part, on the geological category and the petrophysical category of the each sample point; and
generating the virtual model of subterranean region based, at least in part, on the hybrid category assigned to the each sample point within the plurality of sample points.

2. The method of claim 1, wherein generating the virtual model of a subterranean region, further comprises:
obtaining, using a logging unit, at least one well log covering the plurality of sample points in the first wellbore;
training, using the at least one well log and the hybrid category of the each sample point in the plurality of sample points, a machine learning (ML) network to predict a hybrid category of a prediction point from a measured well log containing the prediction point;
obtaining, using the logging unit, at least one well log covering a plurality of prediction points in a second wellbore;
predicting, using the trained ML network, the hybrid category of each prediction point of the plurality of prediction points in the second wellbore from the at least one well log covering the plurality of prediction points; and
generating the virtual model of a subterranean region based, at least in part, on the predicted hybrid category for the each prediction point of the plurality of prediction points.

3. The method of claim 2, wherein the first wellbore comprises the second wellbore.

4. The method of claim 1, wherein obtaining the characteristics of the plurality of sample points, comprises:
collecting, using a core collection unit, a core sample at some of the plurality of sample points; and
for each core sample:
performing a geological examination of the core sample, and
performing, using a petrophysical analyzer, a petrophysical analysis of the core sample.

5. The method of claim 1, wherein the geological category is a modified Dunham Classification assigned based, at least in part, on a grainsize distribution.

6. The method of claim 1, wherein the petrophysical category is assigned based, at least in part, on a pore throat size distribution of Winland R₃₅ method.

7. The method of claim 1, wherein generating the virtual model of a subterranean region based, at least in part, on the hybrid category assigned to the each sample point comprises a digital representation of a three-dimensional distribution of the hybrid category.

8. The method of claim 1, further comprising:
determining a drilling target based, at least in part, on the virtual model of a subterranean region;
planning, using a wellbore planning unit, a planned wellbore trajectory to intersect the drilling target; and
drilling, using a drilling unit, a wellbore guided by the planned wellbore trajectory to produce hydrocarbon.

9. The method of claim 1, assigning a geological category comprising:
formulating a geological model based on classification analysis to predict the geological category of the plurality of sample points;
obtaining, using a logging unit, at least one well log covering a plurality of analysis points in a third wellbore; and
predicting, a geological category for each analysis point of the plurality of analysis points in the third wellbore based, at least in part, on the at least one well log covering the plurality of analysis points.

10. The method of claim 2, wherein the second wellbore is the first wellbore.

11. A system of generating a virtual model of a subterranean region, comprising:
a processor in data communication with a logging unit of a first wellbore that:
obtains characteristics of a plurality of sample points within the first wellbore traversing the subterranean region, by retrieving data about the subterranean region stored in a memory and measurements recorded at the logging unit,
for each sample point within the plurality of sample points:
assigns a geological category based, at least in part, on the characteristics;
assigns a petrophysical category based, at least in part, on the characteristics; and
assigns a hybrid category based, at least in part, on the geological category and the petrophysical category; and
generates the virtual model of a subterranean region based, at least in part, on the hybrid category assigned to the each sample point within the plurality of sample points; and
the memory connected to the processor; and
wherein the virtual model of a subterranean region is displayed as a three-dimensional illustration,
wherein the logging unit records the measurements about the plurality of sample points within the first wellbore.

12. The system of claim 11, further comprising: a logging unit configured to obtain at least one well log covering the plurality of sample points in the first wellbore and at least one well log covering a plurality of prediction points in a second wellbore,
wherein the processor is configured to:
train, using the at least one well log and the hybrid category of the each sample point in the plurality of sample points, a machine learning (ML) network to predict a hybrid category of a prediction point within the plurality of prediction points from the at least one well log covering the plurality of prediction points;
predict, using the trained ML network, the hybrid category of each prediction point of the plurality of prediction points from the at least one well log covering the plurality of prediction points; and
generate the virtual model of a subterranean region based, at least in part, on the predicted hybrid category for the each prediction point of the plurality of prediction points.

13. The system of claim 12, wherein the first wellbore comprises the second wellbore.

14. The system of claim 11, further comprising:
a core collection unit configured to collect a core sample in some of the plurality of sample points; and
a petrophysical analyzer configured to perform a petrophysical analysis of the core sample,
wherein the processor is further configured to perform a geological examination of the core sample.

15. The system of claim 11, wherein the geological category is a modified Dunham Classification assigned based, at least in part, on a grainsize distribution.

16. The system of claim 11, wherein the petrophysical category is assigned based, at least in part, on a pore throat size distribution of Winland R₃₅ method.

17. The system of claim 11, wherein the virtual model of a subterranean region is generated based, at least in part, on the hybrid category assigned to the each sample point and comprises a digital representation of a three-dimensional distribution of the hybrid category.

18. The system of claim 11, further comprising:
a wellbore planning unit configured to plan a planned wellbore trajectory to intersect a drilling target; and
a drilling unit configured to drill a wellbore guided by the planned wellbore trajectory to produce hydrocarbon,
wherein the processor is configured to: determine the drilling target based, at least in part, on the virtual model of a subterranean region.

19. The system of claim 11, further comprising: a logging unit configured to obtain at least one well log covering a plurality of analysis points in a third wellbore,
wherein the processor is configured to:
formulate a geological model based on classification analysis to predict the geological category of the plurality of sample points; and
predict a geological category for each analysis point of the plurality of analysis points based, at least in part, on the at least one well log covering the plurality of analysis points.

20. The system of claim 12, wherein the second wellbore is the first wellbore.
